(19) United States
(12) Patent Application Publication
(10) Pub. No.: US 2023/0165762 A1
(43) Pub. Date: Jun. 1, 2023

(54) FOAMING MAKEUP REMOVER

(71) Applicant: ELEGANT BEST INVESTMENT LIMITED, Causeway Bay, Hong Kong (CN)

(72) Inventors: Xiaodong HE, Auzeville Tolosane (FR); Pauline SAUREL, Vallegue (FR); Julie SAINTECATHERINE, L'Union (FR); Lise MASSON, Toulouse (FR)

(21) Appl. No.: 17/996,771

(22) PCT Filed: Apr. 21, 2021
(86) PCT No.: PCT/IB2021/053277

§ 371 Date: Oct. 20, 2022

(30) Foreign Application Priority Data
Apr. 22, 2020 (FR) 2004024

(51) Int. Cl. A61K 8/06; A61Q 1/14

(52) U.S. Cl. CPC A61K 8/062; A61Q 1/14

(57) ABSTRACT

A cosmetic composition for makeup removal is in the form of an oil-in-water emulsion. The composition includes a lipophilic phase having a content by weight between 35% and 60% with respect to the total weight of the composition. The composition has a non-ionic hydrophilic surfactant assembly with at least one alkylpolyglucoside. The composition further includes at least one gelling agent having a content by weight between 0.3% and 0.8% with respect to the total weight of the composition. The gelling agent is at least one clay and/or at least one polysaccharide gum.

### TECHNICAL FIELD OF THE INVENTION

The present invention relates in particular to a cosmetic composition and the use of this cosmetic composition for keratin material makeup removal.

### TECHNICAL BACKGROUND

Skin makeup removal is an essential step of face care. It must be done daily. It allows to get rid of impurities, pollution and make-up residue.

Among the numerous cosmetic compositions for makeup removal, cleansing foams have the advantage of being gentle and refreshing.

However, it is difficult to formulate makeup removing foams which are both comfortable for the skin and have good makeup removing properties.

Thus, the foaming makeup removers in the form of an oil-in-water emulsion are comfortable on application due to their oily phase, but has very bad foaming properties.

On the contrary, compositions like foaming gels which contain a high foaming surfactant content have good foaming properties, but are more aggressive for the skin than oil-in-water emulsions.

In this context, there is a need for a cosmetic makeup removing composition which has one or more of the following qualities:
an excellent effectiveness to remove both aqueous and anhydrous make-up,
an easy application,
a good capacity to form a foam,
a comfortable application.

### SUMMARY OF THE INVENTION

To meet this need, the inventors have formulated a cosmetic composition in the form of an oil-in-water emulsion with a high percentage of oil which has both good foaming properties and is easy to apply. This cosmetic composition forms a foam which breaks up during makeup removal and allows to remove, both just as well for aqueous make-up and anhydrous make-up.

An aim of the present invention consequently relates to a cosmetic composition for makeup removal in the form of an oil-in-water emulsion comprising:
a lipophilic phase in a content by weight comprised between 35% and 60% with respect to the total weight of the composition,
a non-ionic hydrophilic surfactant assembly comprising at least one alkylpolyglucoside,
at least one gelling agent in a content by weight comprised between 0.3% and 0.8% with respect to the total weight of the composition, the gelling agent being:
at least one clay and/or
at least one polysaccharide gum.

### DETAILED DESCRIPTION OF THE INVENTION

### Definitions

The expression “comprised between . . . and . . . ” must comprise inclusive limits. All throughout the present application and unless it is otherwise specified, the percentages are percentages by weight with respect to the total weight of the composition.

### Cosmetic Composition

The present invention relates to a cosmetic composition such as defined above. Typically, the cosmetic composition according to the invention is foaming. It can be in the form of a one-phase fluid emulsion such as a milk or multi-phase, such as an oil/emulsion two-phase emulsion. It can also be in the form of a foam when a gas, preferably air, is dispersed in this composition.

Lipophilic Phase

As indicated below, the cosmetic composition according to the invention is in the form of an oil-in-water emulsion.

Oil-in-water emulsions comprising a lipophilic phase dispersed in a hydrophilic phase.

The lipophilic phase also called oily or fatty phase, is constituted of lipophilic constituents, i.e. oils and other lipophilic bodies present in the composition, as well as all the lipophilic additive possibly present.

The content by weight of the lipophilic phase is comprised between 35% and 60% with respect to the total weight of the composition. Preferably, the content by weight of the lipophilic phase is comprised between 45% and 55% with respect to the total weight of the composition.

Advantageously, the lipophilic phase contains at least one oil, in particular a cosmetic oil. By “oil”, this means a liquid fat body at ambient temperature.

The oil can be selected from among oils of a mineral, plant origin, or synthetic. The oil can, for example, be selected from the group consisting of an alkane such as paraffin, Vaseline, isohexadecane and isododecane, a fatty ester, a fatty ether or a mixture of these.

According to a preferred embodiment, the oil is a fatty ester or a fatty ester mixture.

Preferably, the composition does not comprise any fatty alcohol.

As a fatty ester, for example CETIOL C5C®, NIKKOL IPIS®, CRODAMOL PTIS™ and LIPONATE NPGC-2® can be cited.

Hydrophilic Phase

Advantageously, the hydrophilic phase comprises water and due to this, can also be called aqueous phase.

Further to water, the hydrophilic phase can also comprise other hydrophilic solvents such as alcohol, for example ethanol or isopropanol.

The hydrophilic phase also comprises hydrophilic compounds other than the hydrophilic solvent(s).

According to a preferred embodiment, the oil/water ratio is comprised between 0.5 and 1.5.

Surfactant

The composition according to the invention comprises a non-ionic hydrophilic surfactant assembly.

According to a preferred embodiment, the content of the surfactant assembly is comprised between 5% and 10% by weight with respect to the total weight of the composition.

Advantageously, the surfactant assembly has a hydrophilic-lipophilic balance (HLB) comprised between 12 and 18, more preferably between 14 and 16.

The hydrophilic-lipophilic balance (HLB) of a surfactant is a measurement of the degree of hydrophilia or of lipophilia well-known to a person skilled in the art (Griffin, William C. (1949), “Classification of Surface-Active Agents by ‘HLB’” (PDF), Journal of the Society of Cosmetic Chemists, 1 (5): 311-26, Griffin, William C. (1954), “Calculation of HLB Values of Non-Ionic Surfactants”, Journal of the Society of Cosmetic Chemists, 5 (4): 249-56, “The HLB system. A time-saving guide to Emulsifier selection” (published by ICI Americas Inc.; 1984).

A surfactant assembly with an HLB comprised between 12 and 18 and more preferably between 14 and 16 allows to obtain a composition with a good capacity to form foam.

The surfactant assembly comprises at least one alkylpolyglucoside. Alkylpolyglucosides are known to a person skilled in the art. They have preferably the formula [C₆H₁₁O₅][C₆H₁₀O₅]ₙOR where R is an alkyl group having 1 to 20 carbon atoms, more preferably from 8 to 14 carbon atoms, and n is an average value of zero or greater than 0.

According to a preferred embodiment, the surfactant assembly does not comprise any amphoteric surfactant.

Gelling Agent

As indicated above, the cosmetic composition according to the invention has a content of gelling agent which is comprised between 0.3% and 0.8% by weight with respect to the total weight of said composition. More preferably, the content of gelling agent is comprised between 0.4% and 0.6%, even more preferably, 0.5% by weight with respect to the total weight of said composition.

Typically, if the concentration of gelling agent is too high, the cosmetic composition will be too viscous and it cannot form foam. If the concentration of gelling agent is too low, the emulsion will be unstable.

The concentration of the gelling agent therefore allows to obtain the desired viscosity for the cosmetic composition which thus has both a good capacity to form foam and to form a stable emulsion.

Thus, preferably, the viscosity of the cosmetic composition according to the invention measured at ambient temperature at t=60 seconds with the measuring device of type RM 100 PLUS LAMY RHEOLOGY is comprised between 200 cP and 400 cP.

The at least one gelling agent of the cosmetic composition according to the invention is at least one clay and/or at least one polysaccharide gum.

Clay can be selected from the group consisting of bentonite, montmorillonite, saponite, hectorite, fluorohectorite, beidellite, nontronite, vermiculite, halloysite, ghassoul, stevensite and a mixture of these. Clay is preferably hectorite, bentonite, montmorillonite and/or a mixture of these. These clays are indeed commonly used as agent for controlling the viscosity in cosmetic products. More preferably, clay is hectorite.

Polysaccharide gum can be selected from the group consisting of xanthan gum, tara gum, carrageenan gum, gellan gum, rhizobian gum, Sclerotium rolfssii gum and a mixture of these. Polysaccharide gum is preferably xanthan gum.

According to a preferred embodiment, the at least one gelling agent of the cosmetic composition is a clay, more preferably hectorite. Thus, the cosmetic composition has a better stability and a better capacity to form foam.

According to a preferred embodiment, the composition according to the invention does not comprise any polymer or copolymer cross-linked with a monomer, cationic or anionic, with ethylenic unsaturation such as carbomers.

Others

The cosmetic composition can further comprise any other compound usable in the cosmetic field.

Thus, the cosmetic composition according to the invention can comprise active ingredients such as, for example, hydrating agents, anti-aging agents, anti-seborrheic agents, preservatives, vitamins, antioxidants, trace elements and/or perfumes.

Foam Dispensing Device

The present invention also relates to a foam dispensing device comprising a tank which comprises the cosmetic composition according to the invention.

Foam dispensing devices are found usually in commerce (WO97/13585, EP0613728). Usually, further to their tank, foam dispensing devices comprise a foam-forming means. The foam-forming means allows the dispersion of a gas in liquid coming from the tank.

In the present case, the foam-dispensing device therefore comprises a foam-forming means to disperse a gas in the cosmetic composition coming from the reservoir.

Preferably, the foam-dispensing device does not comprise any propulsive gas. The foam is formed by a dispersion of air in the cosmetic composition.

### Use

The present invention also relates to the cosmetic use of the cosmetic composition such as defined above as a product for makeup removal.

The present invention also relates to a method for keratin material makeup removing wherein the keratin material is selected from the group consisting of skin, lips, hair, eyelashes and eyebrows, wherein the cosmetic composition such as defined above is applied on the keratin material.

Preferably, the keratin material which is cleansed by the cosmetic composition according to the invention is skin, lips, eyelashes and/or eyebrows.

According to a preferred embodiment, the cosmetic composition according to the invention is a makeup remover. It can be used to remove any type of make-up such as lipstick, foundation, mascara and/or eyeshadow. The cosmetic composition according to the invention has the advantage of being able to remove both aqueous and oily make-up.

The invention will be further illustrated by the FIGURE and the following examples.

However, this FIGURE and these examples must not be interpreted as limiting the scope of the present invention.

### BRIEF DESCRIPTION OF THE FIGURES

FIG. 1 shows the comparison of the cleansing products on an anhydrous-based lipstick (Powermatte Lip Pigment by NARS Cosmetics), on an aqueous-based waterproof mascara (Lash augmented mascara by Nocibé) and on a coloured lip ink (Lip Tattoo by Dior Addict) of a cleansing composition according to an embodiment of the invention versus a commercial makeup remover.

### EXAMPLES

### Material and Methods

Water is placed in a 250 ml beaker, as well as gelling agent, then a gel is formed by stirring the composition for 10 minutes at 600 rotations per minute by means of a TURBOTEST EVO RAYNERI device provided with a grooved stator, this being placed centrally in the beaker.

Then, surfactants are added in the gel obtained then the assembly is homogenised for 5 minutes at 600 rotations per minute using a TURBOTEST EVO RAYNERI device provided with a grooved stator, this being placed centrally in the beaker.

Then, the emulsion is made by pouring, little by little, the fatty phase previously homogenised in the hydrophilic phase stirring at 3300 rotations per minute for 5 minutes still using a TURBOTEST EVO RAYNERI device provided with a grooved stator, this being placed centrally in the beaker.

Finally, the agent is added into the emulsion obtained then the assembly is homogenised for 5 minutes at 3000 rotations per minute using a TURBOTEST EVO RAYNERI device provided with a grooved stator, this being placed centrally in the beaker.

Then, the viscosity of the emulsion obtained is measured at t=60 seconds at ambient temperature using a RM 100 PLUS LAMY RHEOLOGY viscosimeter with the following parameters:
Measuring system MS-R3; speed of 45 rotations per minute for emulsions of which the viscosity is comprised between 72 cP and 21600000 cP
Measuring system MS-DIN 11; speed of 193.65 rotations per minutes for emulsions of which the viscosity is comprised between 3 cP and 1000000 cP Finally, the emulsion obtained is decanted into a foam pump without propulsive gas-type packaging and it is observed if foam is obtained.

If foam is obtained with the composition tested by this method, the foaming properties of the tested composition are considered as acceptable and utilisable.

### Results

### Foaming Properties

TABLE 1

| | | | | | Commercial foaming |
|---|---|---|---|---|---|
| | | | | | gel/cream of which |
| | EXAMPLES | | | | the gelling agent is of |
| | 1 | 2 | 3 | 4 | the polyacrylate type |
| GELLING AGENTS: | | | | | |
| BENTONE EW CE | 0.5% | 0.5% | | | acrylamide/sodium |
| LAPONITE XLG | | | 0.5% | | acryloyldimethyltaurate |
| CARPBOPOL | | | | 0.9% | copolymer |
| ULTREZ 30 | | | | | |
| acrylamide/sodium | | | | | |
| acryloyldimethyltaurate | | | | | |
| copolymer | | | | | |
| SURFACTANTS: | | | | | |
| CREMOPHOR CO 40 | 4% | | | 1% | |
| ORAMIX CG110 | 4% | 4% | 4% | | |
| ORAMIX NS10 | | 4% | 4% | 5% | |
| OILS: | | | | | |
| CETIOL C5C | 12.77% | 12.77% | 12.77% | | |
| NIKKOL IPIS | 5.32% | 5.32% | 5.32% | | |
| CRODAMOL PTIS | 15.96% | 15.96% | 15.96% | | |
| LIPONATE NPGC-2 | 15.96% | 15.96% | 15.96% | | |
| TEGOSOFT CT | | | | 50% | |
| AGENT: | | | | | |
| PHYTOFOAM ™ | 2% | 2% | 2% | | |
| Water QS | | | | | |
| Obtaining of foam | yes | yes | yes | no | no |

The commercial products used in these three examples have the following INCI names:

KELTROL CG-F: Xanthan gum

BENTONE EW CE: Hectorite

LAPONITE XLG: Lithium magnesium sodium silicate

CREMOPHOR CO 40: PEG-40 hydrogenated castor oil

ORAMIX CG110: Caprylyl/capryl glucoside

ORAMIX NS10: Decyl glucoside

CETIOL C5C: Coco-caprylate/caprate

NIKKOL IPIS: Isopropyl isostearate

CRODAMOL PTIS: Pentaerythrityl Tetraisostearate

LIPONATE NPGC-2: Neopentylglycol dicaprylate/dicaprate

PHYTOFOAM: Aqua (and) Acacia Concinna Fruit Extract (and) Balanites

Aegyptiaca Fruit Extract (and) Gypsophila Paniculata Root Extract

The compositions of examples 1 to 3 have satisfactory foaming properties, contrary to the composition of example 4 and to the commercial makeup remover foaming gel/cream with a polyacrylate-type gelling agent.

### Effect of the Viscosity on the Production of Foam

The link between viscosity and foam production is studied from different emulsions containing different percentages of one same gelling agent.

The tested compositions have, as a formulation:
8.0% of a surfactant assembly of which the average HLB is comprised between 12 and 16 and comprising at least one glucoside-type surfactant
variable concentrations of a clay- or xanthan-type gelling agent
50% of an oily mixture comprising cleansing or hydrating oils
water (QS)

The percentages of the gelling agents used are: 0%; 0.2%; 0.5%; 0.8% and 1%.

The results are summarised in the following table:

TABLE 2

| % of gelling | | |
|---|---|---|
| agent in the | Viscosity | Production |
| tested composition | (cP) | of foam |
| 0 | 41.67 | yes |
| 0.2 | 105.5 | yes |
| 0.5 | 232.4 | yes |
| 0.8 | 297.0 | yes |
| 1 | 423.9 | no |

The results obtained show that the tests of which the viscosity is greater than around 400 cP do not allow to obtain a foam.

### Cleansing Properties

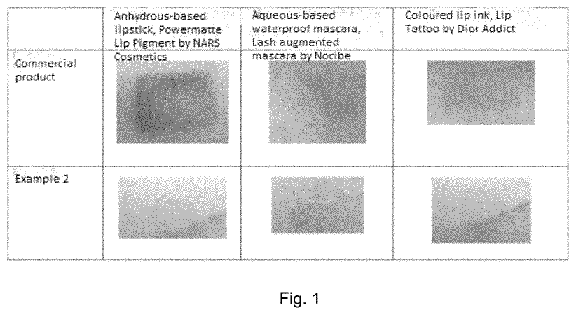

The cleansing properties of the composition of example 2 are compared to those of a commercial cleanser having a content by weight of oily phase less than 20% (FIG. 1).

The composition of example 2 has shown better cleansing properties both on an anhydrous-based lipstick, on an aqueous-based waterproof mascara and on a coloured lip ink.

1. A cosmetic composition in the form of an oil-in-water emulsion, which comprises:
a lipophilic phase in a content by weight comprised between 35% and 60% with respect to a total weight of the composition,
a non-ionic hydrophilic surfactant assembly comprising at least one alkylpolyglucoside,
at least one gelling agent in a content by weight comprised between 0.3% and 0.8% with respect to the total weight of the composition, the gelling agent comprising:
at least one clay and/or
at least one polysaccharide gum.

2. The cosmetic composition according to claim 1, wherein the gelling agent is a clay.

3. The cosmetic composition according to claim 1, wherein the surfactant assembly has a hydrophilic-lipophilic balance (HLB) comprised between 12 and 18.

4. The cosmetic composition according to claim 1, wherein the content by weight of the surfactant assembly is between 5% and 10% with respect to the total weight of the composition.

5. The cosmetic composition according to claim 1, wherein the oil/water ratio is between 0.5 and 1.5.

6. The cosmetic composition according to claim 1, wherein the content by weight of the lipophilic phase is between 45% and 55% with respect to the total weight of the composition.

7. The cosmetic composition according to claim 1, wherein the cosmetic composition is a makeup remover.

8. A form-dispensing device comprising a tank which comprises the cosmetic composition according to claim 1.

9. A method of using the cosmetic composition according to claim 1, comprising removing makeup with the cosmetic composition.

10. A method for cleansing a keratin material, comprising applying the cosmetic composition according to claim 1 onto the keratin material.
